(19) United States
(12) Patent Application Publication
(10) Pub. No.: US 2012/0197211 A1
(43) Pub. Date: Aug. 2, 2012

(54) MEDICINE DISPENSER HAVING A THREADED PLUNGER

(76) Inventors: Ward Brister, Grenada, MS (US); Henry Brister, Grenada, MS (US); Larry Watson, Grenada, MS (US)

(21) Appl. No.: 13/019,055

(22) Filed: Feb. 1, 2011

(51) Int. Cl. A61M 5/31

(52) U.S. Cl. 604/207

(57) ABSTRACT

A medicine dispenser includes a barrel to store medicine, a slidable shuttle disposed in the barrel, a threaded shaft including a threaded outer surface and a trench formed along the threaded outer surface, and a threaded cap connected to the barrel and including an opening through which the threaded shaft is threaded into, and a feedback member protruding inside the opening, wherein the threaded shaft pushes the slidable shuttle to dispense the medicine out of the barrel and the feedback member interlocks into the trench according to a rotation of the threaded shaft.

### FIELD OF THE INVENTION

Aspects of the present invention relate to a medicine dispenser, and more particularly, aspects of the present invention relate to a medicine dispenser which can accurately dispense low doses of medicine while alerting the user to the amount of medicine which has been dispensed.

### BACKGROUND OF THE RELATED ART

Medicine dispensers used to dispense creams, gels, ointments, liquids and gases are commonly employed in many different environments, including hospitals, nursing homes, and private residences. Conventionally, medicine dispensers have been used which only work with relatively large doses of medicine. For example, conventional medicine dispensers are only capable of accurately dispensing doses of medicine well above 0.1 milliliter (mL). However, given that many different types of medicines should be administered in doses of 0.1 mL or below, these conventional medicine dispensers are not sufficient.

Furthermore, many of the people who use medicine dispensers are elderly, and may be hearing impaired and have bad vision or other ailments. However, the conventional medicine dispensers do not indicate to the user how much medicine has been dispensed, or to the extent that an indication is given to the user, the indication may not be sufficient to alert certain people. For example, although the conventional medicine dispenser may have measurement markings on the side of the dispenser, a patient who has bad vision may not be able to read the measurement markings in a reliable fashion. Thus, many of the people who use conventional medicine dispensers do not have an effective way of monitoring how much medicine has been dispensed, which can in turn lead to improper medicine dosages.

Moreover, conventional medicine dispensers for liquids, gels, etc. often come in the form of syringes which are discarded after use. However, these syringes are often inconvenient to carry, are not reusable, and are not child-proof.

Thus, there is a need for a medicine dispenser which can accurately dispense extremely low dosages (0.1 mL and below) of medicine, such as creams, gels, ointments, liquids, gases and other types of medicines.

There is further a need for a medicine dispenser which effectively alerts the user to the amount of medicine which has been dispensed.

There is further a need for a medicine dispenser which is convenient to carry and use, reusable, and safe to keep in the house.

### SUMMARY OF THE INVENTION

Aspects of the present invention solve these and other problems by providing a medicine dispenser which can very accurately dispense extremely low dosages (0.1 mL and below) of medicine using a simple and reliable design.

Aspects of the present invention further provide a medicine dispenser which effectively alerts the user to the amount of medicine which has been dispensed by providing both tactile and auditory feedback to the user.

Aspects of the present invention further provide a medicine dispenser which is disposable and/or reusable.

Aspects of the present invention further provide a medicine dispenser having a mechanism which cannot be accidentally deployed, making the medicine dispenser extremely safe and child-resistant.

Aspects of the present invention further provide a medicine dispenser which has a configuration that is conventional for travel.

Aspects of the present invention further provide a medicine dispenser which is configured as a single container instead of multiple syringes.

According to an aspect of the present invention, a medicine dispenser includes a barrel to store medicine, a slidable shuttle disposed in the barrel, a threaded shaft including a threaded outer surface and a trench formed along the threaded outer surface, and a threaded cap connected to the barrel and including an opening through which the threaded shaft is threaded into, and a feedback member protruding inside the opening, wherein the threaded shaft pushes the slidable shuttle to dispense the medicine out of the barrel and the feedback member interlocks into the trench according to a rotation of the threaded shaft.

According to an aspect, the threaded shaft includes a plurality of the trenches and the threaded cap includes a plurality of the feedback members.

According to an aspect, the threaded shaft includes four of the trenches spaced apart from each other at 90 degrees and two of the feedback members spaced 180 degrees apart from each other in the threaded cap.

According to an aspect, the barrel has an inner diameter of approximately 22.5 mm and the threaded outer surface of the threaded shaft comprises threads spaced apart from each other by 1 mm, so that each 90 degree rotation of the threaded shaft dispenses 0.1 mL of medicine.

According to an aspect, the medicine dispenser further includes a nozzle on one end of the barrel, a plug to plug the nozzle, and a knob on one end of the threaded shaft opposite the end which is inserted into the threaded cap, wherein the threaded shaft is rotated by rotating the knob.

According to an aspect, the feedback member is configured as a protrusion formed in the threaded cap and generates tactile and audible feedback when the trench interlocks with the feedback member.

According to an aspect, the threaded shaft is 12 mm in diameter.

According to an aspect, the medicine is one of a cream, gel, ointment, liquid, or gas.

According to another aspect of the present invention, a medicine dispenser includes a barrel to store medicine, and a shaft including a trench formed along an axis of the outer surface, wherein an end of the shaft dispenses the medicine out of the barrel and the trench interlocks into a feedback member protruding inside the barrel according to a rotation of the shaft.

According to another aspect, the medicine dispenser includes a slidable shuttle disposed in the barrel, wherein the end of the shaft dispenses the medicine by contacting and pressing the slidable shuttle according to the rotation.

According to another aspect, the shaft is a threaded shaft and is threaded into the barrel.

According to another aspect, the feedback member is configured as a protrusion and generates tactile and audible feedback when the trench interlocks with the feedback member.

According to yet another aspect, a method to dispense medicine includes storing medicine in a barrel, the barrel comprising a slidable shuttle disposed in the barrel, threading a threaded shaft including a threaded outer surface and a trench formed along the threaded outer surface into a threaded cap connected to the barrel, the threaded cap including an opening through which the threaded shaft is threaded into, and a feedback member protruding inside the opening, and rotating the threaded shaft to push the slidable shuttle and thereby dispense the medicine out of the barrel and to interlock the feedback member into the trench.

According to yet another aspect, the threaded shaft includes a plurality of the trenches and the threaded cap includes a plurality of the feedback members.

According to yet another aspect, the threaded shaft includes four of the trenches spaced apart from each other at 90 degrees and two of the feedback members spaced 180 degrees apart from each other in the threaded cap.

According to yet another aspect, the barrel has an inner diameter of approximately 22.5 mm and the threaded outer surface of the threaded shaft includes threads spaced apart from each other by 1 mm, so that each 90 degree rotation of the threaded shaft dispenses 0.1 mL of medicine.

According to yet another aspect, the medicine is delivered into and dispensed out of the barrel through a nozzle on one end of the barrel, and the threaded shaft includes a knob disposed opposite the end which is inserted into the threaded cap, wherein the rotating of the threaded shaft includes rotating the knob.

According to yet another aspect, the feedback member is configured as a protrusion formed in the threaded cap and generates tactile and audible feedback when the trench interlocks with the feedback member.

According to yet another aspect, the threaded shaft is 12 mm in diameter.

According to yet another aspect, the medicine is one of a cream, gel, ointment, liquid, or gas.

### BRIEF DESCRIPTION OF THE FIGURES

The accompanying drawings, which are incorporated in and constitute a part of the specification, illustrate presently preferred non-limiting examples of exemplary embodiments of the invention, and, together with the general description given above and the detailed description of the preferred embodiments given below, serve to explain the principles and concepts of the invention, in which like reference characters designate like or corresponding parts throughout the several drawings. Preferred embodiments of the present invention will now be further described in the following paragraphs of the specification and may be better understood when read in conjunction with the attached drawings, in which:

FIG. 1 depicts a side view of a medicine dispenser according to an embodiment of the present invention;

FIG. 2 depicts a sectioned end view of the threaded cap shown in FIG. 1; and

FIG. 3 depicts a magnified view of a portion of the sectioned end view shown in FIG. 2.

### DETAILED DESCRIPTION OF THE PREFERRED EMBODIMENTS

Reference will now be made in detail to the presently non-limiting, exemplary and preferred embodiments of the invention as illustrated in the accompanying drawings. The nature, concepts, objectives and advantages of the present invention will become more apparent to those skilled in the art after considering the following detailed description in connection with the accompanying drawings. The following description is provided in order to explain preferred embodiments of the present invention, with the particular features and details shown therein being by way of non-limiting illustrative examples of various embodiments of the present invention. The particular features and details are presented with the goal of providing what is believed to be the most useful and readily understood description of the principles and conceptual versions of the present invention. In this regard, no attempt is made to show structural details of the invention in more detail than is necessary for the fundamental understanding of the present invention. The detailed description considered with the appended drawings are intended to make apparent to those skilled in the art how the several forms of the present invention may be embodied in practice.

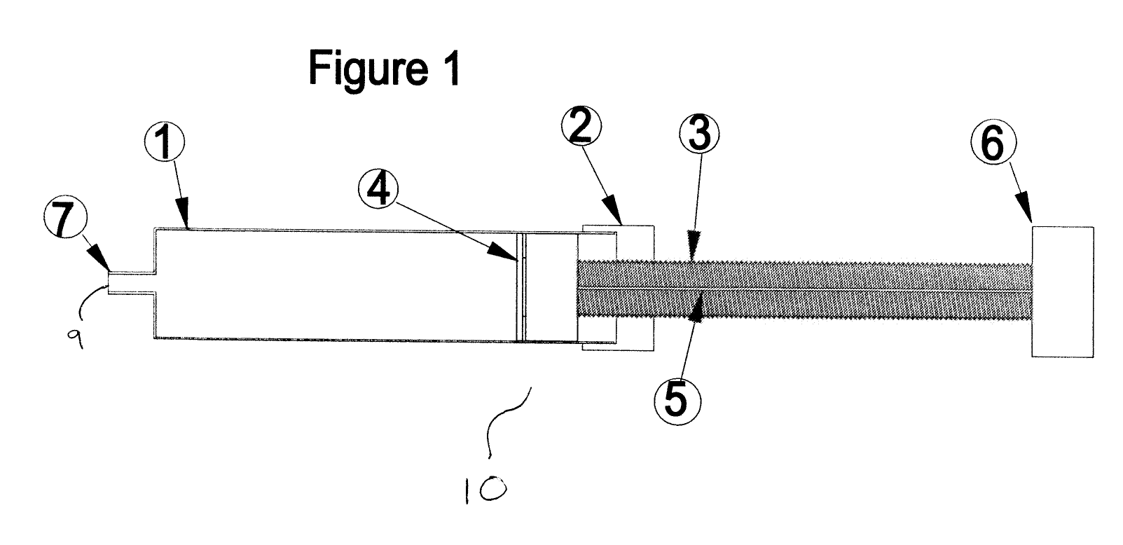

FIG. 1 depicts a side view of a medicine dispenser according to an embodiment of the present invention. As shown in FIG. 1, the medicine dispenser 10 includes a barrel 1 to store medicine and a detachable threaded shaft 3, i.e., a threaded plunger, which presses medicine out of the barrel as it is rotated, to accurately dispense medicine to patients. The barrel 1 and detachable threaded shaft 3 may be made of various materials, such as plastic, glass, or a combination thereof.

The barrel 1 includes a nozzle 7 through which the medicine is transferred on one end, and a shuttle 4 which is disposed inside of the barrel 1 at another end opposite the end with the nozzle 7. As shown in FIG. 1, the shuttle 4 is a flat, circular shaped surface which is slidable along the length of the barrel 1 according to the movement of the threaded screw 3 and has a diameter which is approximately equal to the inner diameter of the barrel 1 to form a snug fit. The shuttle 4 can be configured to slide along the inside of the barrel 1 in various ways known to those of skill in the art, such as by using tracks, grooves, etc. on the shuttle 4 and inner surface of the barrel 1. As the shuttle 4 is moved along the inside of the barrel 1 towards the nozzle 7, medicine is dispensed out of the nozzle 7. The outer surface of the barrel 1 at the end opposite the nozzle 7 is threaded. The barrel 1 further includes a plug 9 to plug the nozzle 7 to prevent a loss of medicine when the medicine dispenser 10 is not in use. The barrel 1 can store various different types of medicine, including creams, gels, ointments, liquids, and gases.

The threaded shaft 3 includes a threaded cap 2 disposed at one end which has an opening that the threaded shaft 3 is threaded into, and four trenches 5 cut in a direction parallel to the long axis of the threaded shaft 3 (in the left-right direction of FIG. 1) and spaced 90 degrees apart from each along the outer circumference of the threaded shaft 3. The threaded shaft 3 further includes a knob 6 to twist the threaded shaft 3 into the threaded cap 2. As shown in FIG. 1, the threaded cap 2 has two sets of threads: an outer set of threads to thread the threaded cap 2 onto the corresponding threads on the outer surface of the barrel 1, and an inner set of threads which are threaded with the threaded shaft 3 and permit the threaded shaft 3 to move through the threaded cap 2. Once the threaded cap 2 is threaded to the barrel 1 and the threaded shaft 3 is threaded into the threaded cap 2, twisting of the knob 6 causes the end of the threaded shaft 3 to contact the shuttle 4 and press the shuttle 4 along the inside of the barrel 1. Specifically, as the knob 6 is twisted in a clockwise direction, the threaded shaft 3 moves to the left in FIG. 1, contacts the shuttle 4 and moves the shuttle 4 to the left so as to decrease the volume of available space in the barrel 1, thereby ejecting the medication stored in the barrel 1 out of the nozzle 7. Conversely, when the knob 6 is twisted in a counterclockwise direction, the threaded shaft 3 moves to the right as shown in FIG. 1, and the volume of the available space in the barrel 1 is increased. It is understood by those skilled in the art that the threaded shaft 3 and threaded cap 2 can alternately be designed to twist clockwise to increase volume and counterclockwise to decrease volume.

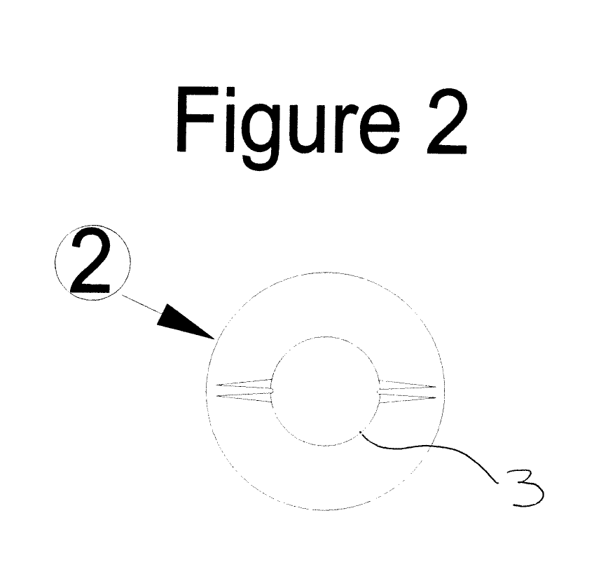

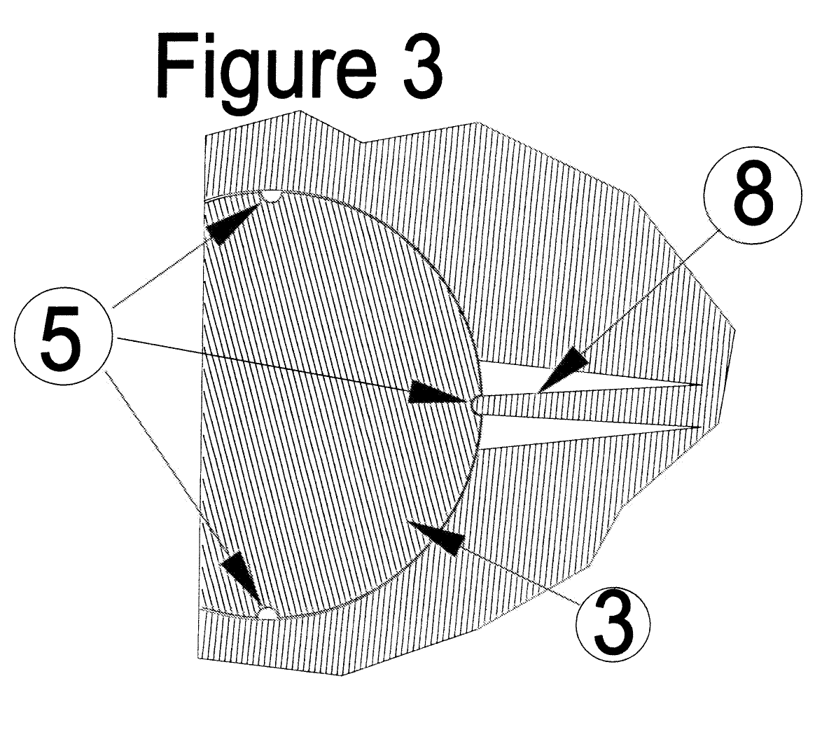

FIG. 2 depicts a sectioned end view of the threaded cap shown in FIG. 1 with the threaded shaft 3 inserted, and FIG. 3 depicts a magnified view of a portion of the sectioned end view shown in FIG. 2 showing the trenches 5 cut in the threaded shaft 3. Also shown in FIG. 3 is one of the rigid feedback fingers 8 molded into the threaded cap 2. The rigid feedback fingers 8 (also referred to as “feedback members”) interact with the trenches 5 of the threaded shaft 3 to generate audible feedback to the patient. Additionally, the feedback fingers 8 have a sufficient level of rigidity to provide a vibration with sufficient power to be felt by the patient when the feedback fingers 8 snap into, i.e., interlock with, the trenches 5.

When the knob 6 is rotated, the threaded shaft 3 connected to the knob 6 also rotates. As the threaded shaft 3 rotates into a position where the trenches 5 interlock with the rigid feedback fingers 8, the rigid feedback fingers produce an audible clicking sound and a vibration. Since four trenches 5 are cut into the threaded shaft 3 in 90 degree intervals along the outer circumference, as shown in FIG. 3, the threaded shaft 3 produces a loud audible clicking sound and a vibration every time the threaded shaft 3 is rotated 90 degrees. Also, according to an embodiment of the present invention, the threaded cap 2 has two rigid feedback fingers 8 spaced opposite each other (180 degrees apart), so that whenever one of the rigid feedback fingers 8 locks into one of the trenches 5, the other rigid feedback finger 8 locks into another one of the trenches 5 located on the other side of the threaded screw 3. It is understood by those skilled in the art, however, that more or less than two rigid feedback fingers 8 may be used in accordance with other aspects of the invention. After interlocking, if the user resumes rotating the knob 6, the threaded screw 3 is correspondingly rotated, and the rigid feedback fingers 8 are unlocked from the trenches 5, until the knob 6 is rotated another 90 degrees. In this fashion, the user is alerted each time that the threaded screw 3 is rotated 90 degrees, thereby providing a very accurate way to dispense medicine.

Data shows that a tubular dispensing chamber, such as the barrel 1, with an inside diameter of 22.5679 mm (0.8885 inches), coupled with the movement of a matching plunger, such as the shuttle 4, of 0.25 mm will result in a change of 100.0028063 cubic mL (0.1000028063 mL) in the volume of the chamber. A chosen method of eliminating human error when dispensing a fluid medication is incorporated by using a defined helical thread combined with the aforementioned volume chamber. According to an aspect of the present invention, the helical thread of the threaded shaft 3 is referred to as M12×1. This thread designation refers to the threaded shaft 3 being 12 mm in diameter and the threads being 1 mm apart (or equivalently, the “lead” of the threads is 1 mm). Since 30,000 cubic millimeters is equal to 30 milliliters, the shuttle 4 should move 1 mm in order to decrease the volume of the barrel 1 by 400 cubic millimeters (which is equal to 0.4 mL). Calculations indicate that a chamber diameter of 22.55 mm (0.888 inches) will achieve this goal. The calculations reveal that a movement of 1 mm will reduce the volume of the barrel 1 by 399.16 cubic millimeters. It follows that a movement of 0.25 mm will result in a reduction of the barrel volume of 99.79 cubic millimeters (0.09979 milliliters). From these statements, it is evident that the medicine dispenser 10 will be at least 99.79% accurate within these broadened dimensional criteria.

In the medicine dispenser 10, the diameter of the barrel 1 is 22.55 mm (0.888 inches). Therefore, advancing the threaded shaft 3 one complete revolution (360 degrees) through the threaded cap 2 moves the plunger exactly 1 mm (0.039 inches). This results in reducing the enclosed volume of the barrel 1 by 0.4 mL (400 cubic millimeters). Therefore, by forming the four trenches 5 parallel to the long axis of the threaded shaft 3 and spaced apart from each other at 90 degree angles, and by further employing the feedback fingers 8, the medicine dispenser 10 achieves tactile and auditory confirmation of an exact rotation of 90 degrees, movement by the plunger of 0.025 mm, and a reduction in the chamber volume of 0.1 mL. Thus, the medicine dispenser 10 according to aspects of the present invention is remarkably accurate for dispensing extremely low dosages (e.g., 0.1 mL) of medicine using a simple and reliable design. Furthermore, the medicine dispenser 10 generates both tactile and auditory feedback to the user in the form of a loud “clicking” sound and a vibration, which is a very reliable way to alert patients each time 0.1 mL of medicine is dispensed. Moreover, since the feedback fingers 8 catch the trenches 5 each time the threaded shaft is rotated 90 degrees, the feedback fingers 8 and trenches 5 help to stabilize and secure the medicine dispenser 10 during use.

A method of using the medicine dispenser 10 will now be described. In this description, it is assumed that the barrel 1 and threaded shaft 3 are initially detached from each other, and that a pharmacist will be delivering the filled and assembled medicine dispenser 10 to a patient upon payment, however, it is understood that the medicine dispenser 10 may be used in other situations as well.

First, a description of filling the medicine dispenser 10 for shipment to a patient is provided. Initially, a medicine, such as a cream, a gel, an ointment, a liquid, or a gas, is delivered into the barrel 1 by attaching a medicine supply to the nozzle 7. The medicine supply can be, for example, a syringe containing the medicine. In order to seal the medicine dispenser 10 for shipment, after the barrel 1 is filled with the medicine, the threaded shaft 3 is connected to the barrel 1 by threading the threaded cap 2 onto threads formed on the outer surface of the barrel 1 and then threading the threaded shaft 3 into the threaded cap 2. The knob 6 is then rotated such that the end of the threaded shaft 3 contacts the shuttle 4, moving the shuttle 4 by rotation of the knob 6 to decrease the volume of medication contained in the barrel 1 and thereby ejecting some of the medication into the nozzle 7. The nozzle 7 is then plugged using the plug 9 by the pharmacist to prevent accidental loss of the medication during shipment.

Next, a description of a patient using the medicine dispenser 10 is provided. Upon receiving the filled and sealed medicine dispenser 10, the patient removes the plug 9 from the nozzle 7, grasps the knob 6, and rotates the knob 6 in a clock-wise direction. When the patient has rotated the knob 6 (and therefore, the threaded shaft 3) 90 degrees, the medicine dispenser 10 will generate a loud “click” and a vibration will be felt by the patient, indicating that 0.1 mL of medication has been ejected from the end of the nozzle 7. Therefore, for instance, if the patient has been directed to use 1.2 mL of medication per day, the patient merely has to rotate the knob 6 until 12 clicks have been heard and felt. After the medication has been dispensed, the patient simply returns the plug 9 to the end of the nozzle 7 and returns the medicine dispenser 10 to storage until next needed.

The above-described method thereby provides a very accurate and reliable way to dispense extremely low dosages (0.1 mL and below) of medicine, while also alerting the user to the amount of medicine which has been dispensed by providing both tactile and auditory feedback to the user.

It is further understood that similar results can be achieved for a complete range of dispenser sizes. For example, an acceptably accurate 5 mL dispenser could be developed by using a tubular chamber with an inside diameter of 11.277 mm (0.444 inches), and would dispense 99.89 cubic millimeters (0.09989 mL) of fluid when the appropriately matched plunger is moved 1 mm. It follows that this arrangement would result in an accuracy of 99.89%. The stem of the accompanying shuttle 4 would probably be sized at M8×1, but would not be limited thereto. Generally, the dispenser according to aspects of the present invention could be configured to eject more or less than 0.1 mL of medicine by adjusting the sizes of various components.

It is further understood that this configuration of the medicine dispenser 10 according to aspects of the present invention can be used as a dispensing mechanism for fluids of almost any viscosity. High viscosity fluids could be dispensed, because tremendous force can be generated through the use of the helical thread design. If appropriate valving and sealants are employed, this configuration also accurately dispenses gaseous medicine.

It is further understood that the medicine dispenser 10 is not limited to having four trenches spaced apart from each other at 90 degrees, but could instead have more or less than four trenches spaced apart from each other more or less than 90 degrees. For example, the threaded shaft 3 could have eight trenches spaced apart from each other at 45 degrees, two trenches spaced apart from each other at 180 degrees, etc. Moreover, the medicine dispenser 10 is not limited to having two feedback fingers 8, but could have more or less than two feedback fingers, and the feedback fingers 8 are also not limited to being shaped as shown in FIG. 3, but could be formed in various different shapes and sizes.

It is further understood that the threaded cap 2 is not limited to connecting to the barrel 1 via threads, and may instead connect to the barrel 1 using various other fastening devices known in the art. Also, according to other aspects, the threaded cap 2 is omitted, and the threaded shaft 3 is threaded into the barrel 1 directly or by some other type of fastening device.

The foregoing description illustrates and describes embodiments of the present invention. However, the disclosure shows and describes only the preferred embodiments of the invention, but it is to be understood that the invention is capable of use in various other combinations, modifications, and environments. Also, the invention is capable of change or modification, within the scope of the inventive concept, as expressed herein, that is commensurate with the above teachings and the skill or knowledge of one skilled in the relevant art. For example, one or more elements of each embodiment may be omitted or incorporated into the other embodiments.

The foregoing implementations and embodiments of the invention have been presented for purposes of non-limiting illustration and description. Although the present invention has been described herein with reference to particular structures, materials and embodiments, the present invention is not intended to be limited to the particular features and details disclosed herein. Rather, the present invention extends to all functionally equivalent structures, methods and uses, such as are within the scope of the appended claims. The descriptions provided herein are not exhaustive and do not limit the invention to the precise forms disclosed. The foregoing embodiment examples have been provided merely for purposes of explanation and are in no way to be construed as limiting the scope of the present invention. The words that have been used herein are words of description and illustration, rather than words of limitation. The present teachings can readily be realized and applied to other types of apparati. Further, modifications and variations, within the purview, scope and spirit of the appended claims and their equivalents, as presently stated and as amended hereafter, are possible in light of the above teachings or may be acquired from practicing the invention. Furthermore, although elements of the invention may be described or claimed in the singular, the plural is contemplated unless limitation to the singular is explicitly stated. Alternative structures discussed for the purpose of highlighting the invention's advantages do not constitute prior art unless expressly so identified. No one or more features of the present invention are necessary or critical unless otherwise specified.

What is claimed is:

1. A medicine dispenser, comprising:
a barrel to store medicine;
a slidable shuttle disposed in the barrel;
a threaded shaft comprising a threaded outer surface and a trench formed along the threaded outer surface; and
a threaded cap connected to the barrel and comprising an opening through which the threaded shaft is threaded into, and a feedback member protruding inside the opening,
wherein the threaded shaft pushes the slidable shuttle to dispense the medicine out of the barrel and the feedback member interlocks into the trench according to a rotation of the threaded shaft.

2. The medicine dispenser of claim 1, wherein the threaded shaft comprises a plurality of the trenches and the threaded cap comprises a plurality of the feedback members.

3. The medicine dispenser of claim 1, wherein the threaded shaft comprises four of the trenches spaced apart from each other at 90 degrees and two of the feedback members spaced 180 degrees apart from each other in the threaded cap.

4. The medicine dispenser of claim 4, wherein the barrel has an inner diameter of approximately 22.5 mm and the threaded outer surface of the threaded shaft comprises threads spaced apart from each other by 1 mm, so that each 90 degree rotation of the threaded shaft dispenses 0.1 mL of medicine.

5. The medicine dispenser of claim 1, further comprising:
a nozzle on one end of the barrel;
a plug to plug the nozzle; and
a knob on one end of the threaded shaft opposite the end which is inserted into the threaded cap,
wherein the threaded shaft is rotated by rotating the knob.

6. The medicine dispenser of claim 1, wherein the feedback member is configured as a protrusion formed in the threaded cap and generates tactile and audible feedback when the trench interlocks with the feedback member.

7. The medicine dispenser of claim 1, wherein the threaded shaft is 12 mm in diameter.

8. The medicine dispenser of claim 1, wherein the medicine is one of a cream, gel, ointment, liquid, or gas.

9. A medicine dispenser, comprising:
a barrel to store medicine; and
a shaft comprising a trench formed along an axis of the outer surface;
wherein an end of the shaft dispenses the medicine out of the barrel and the trench interlocks into a feedback member protruding inside the barrel according to a rotation of the shaft.

10. The medicine dispenser of claim 9, further comprising a slidable shuttle disposed in the barrel, wherein the end of the shaft dispenses the medicine by contacting and pressing the slidable shuttle according to the rotation.

11. The medicine dispenser of claim 10, wherein the shaft comprises a threaded shaft and is threaded into the barrel.

12. The medicine dispenser of claim 11, wherein the feedback member is configured as a protrusion and generates tactile and audible feedback when the trench interlocks with the feedback member.

13. A method to dispense medicine, comprising:
storing medicine in a barrel, the barrel comprising a slidable shuttle disposed in the barrel;
threading a threaded shaft comprising a threaded outer surface and a trench formed along the threaded outer surface into a threaded cap connected to the barrel, the threaded cap comprising an opening through which the threaded shaft is threaded into, and a feedback member protruding inside the opening; and
rotating the threaded shaft to push the slidable shuttle and thereby dispense the medicine out of the barrel and to interlock the feedback member into the trench.

14. The method of claim 13, wherein the threaded shaft comprises a plurality of the trenches and the threaded cap comprises a plurality of the feedback members.

15. The method of claim 13, wherein the threaded shaft comprises four of the trenches spaced apart from each other at 90 degrees and two of the feedback members spaced 180 degrees apart from each other in the threaded cap.

16. The method of claim 15, wherein the barrel has an inner diameter of approximately 22.5 mm and the threaded outer surface of the threaded shaft comprises threads spaced apart from each other by 1 mm, so that each 90 degree rotation of the threaded shaft dispenses 0.1 mL of medicine.

17. The method of claim 13, wherein the medicine is delivered into and dispensed out of the barrel through a nozzle on one end of the barrel, and the threaded shaft comprises a knob disposed opposite the end which is inserted into the threaded cap, wherein the rotating of the threaded shaft comprises rotating the knob.

18. The method of claim 13, wherein the feedback member is configured as a protrusion formed in the threaded cap and generates tactile and audible feedback when the trench interlocks with the feedback member.

19. The method of claim 13, wherein the threaded shaft is 12 mm in diameter.

20. The method of claim 13, wherein the medicine is one of a cream, gel, ointment, liquid, or gas.
